(19) United States
(12) Patent Application Publication
(10) Pub. No.: US 2015/0320601 A1
(43) Pub. Date: Nov. 12, 2015

(54) METHOD AND SYSTEM FOR MEDIATED REALITY WELDING

(71) Applicant: PRISM TECHNOLOGIES LLC, Omaha, NE (US)

(72) Inventor: Richard L. Gregg, Omaha, NE (US)

(73) Assignee: PRISM TECHNOLOGIES LLC

(21) Appl. No.: 14/704,562

(22) Filed: May 5, 2015

Related U.S. Application Data
(60) Provisional application No. 61/989,636, filed on May 7, 2014.

(51) Int. Cl. A61F 9/06; G06T 7/00; G02B 27/01; G06T 1/00; A42B 3/04; A42B 3/22

(52) U.S. Cl. CPC A61F 9/06; A42B 3/042; A42B 3/225; G02B 27/017; G06T 1/0007; G06T 7/0079; G06T 2207/20144

(57) ABSTRACT

A method and system for mediated reality welding is provided. The method and system improves operator or machine vision during a welding operation.

The present application claims the benefit of Provisional Application No. 61/989,636, filed May 7, 2014, the contents of which are incorporated by reference.

### BACKGROUND OF THE INVENTION

1. Field of the Invention

The present invention generally relates to the use of mediated reality to improve operator vision during welding operations. Mediated reality refers to a general framework for artificial modification of human perception by way of devices for augmenting, deliberately diminishing, and, more generally, for otherwise altering sensory input. Wearable computing is the study or practice of inventing, designing, building, or using body-borne computational and sensory devices. Wearable computers may be worn under, over, or in clothing, or may also be themselves clothes. Mediated reality techniques can be used to create wearable computing applications. The promise of wearable computing has the ability to fundamentally improve the quality of our lives.

2. Description of the Prior Art

Eye injuries account for one-quarter of all welding injuries, making them by far the most common injury for welders, according to research from the Liberty Mutual Research Institute for Safety. All of the most common types of welding (shielded metal-arc welding, stick welding, or gas welding) produce potentially harmful ultraviolet, infrared, and visible spectrum radiation. Damage from ultraviolet light can occur very quickly. Normally absorbed in the cornea and lens of the eye, ultraviolet radiation (UVR) often causes arc eye or arc flash, a very painful but seldom permanent injury that is characterized by eye swelling, tearing, and pain. The best way to control eye injuries is also the most simple: proper selection and use of eye protection offered by a welding helmet.

Welding helmets can be fixed shade or variable shade. Typically, fixed shade helmets are best for daily jobs that require the same type of welding at the same current levels, and variable helmets are best for workers with variable welding tasks. Helmet shades come in a range of darkness levels, rated from 9 to 14 with 14 being darkest, which adjust manually or automatically, depending on the helmet. To determine the best helmet for the job, a lens shade should be selected that provides comfortable and accurate viewing of the “puddle” to ensure a quality weld. Integral to the welding helmet is an auto-darkening cartridge that provides eye protection through the use of shade control.

The modern welding helmet used today was first introduced by Wilson products in 1937 using a fixed shade. The current auto-darkening helmet technology was submitted to the United States Patent Office on Dec. 26, 1973 by Mark Gordon. U.S. Pat. No. 3,873,804, entitled “Welding Helmet with Eye Piece Control, issued Mar. 25, 1975 to Gordon and disclosed a LCD electronic shutter that darkens automatically when sensors detect the bright welding arc.

With the introduction of the electronic auto-darkening helmets, the welder no longer had to get ready to weld and then nod their head to lower the helmet over their face. However, these electronic auto-darkening helmets don't help the wearer see better than traditional fixed-shade “glass” during the actual welding. While the welding arc is on, the “glass” is darkened just as it would be if it were fixed-shade, so the primary advantage is the ability to see better the instant before or after the arc is on. In 1981, a Swedish manufacturer named Hornell introduced Speedglas, the first real commercial implementation of Gordon's patent. Since 1981, there have been limited advancements in the technology used to improve the sight of an operator during welding. The auto-darkening helmet remains today as the most popular choice for eye protection.

### SUMMARY OF THE INVENTION

The present invention in a preferred embodiment contemplates a method and system for mediated reality welding by altering visual perception during a welding operation, including obtaining a current image; determining a background reference image; determining a foreground reference image; processing the current image by: (i) combining the current image and the background reference image, and (ii) substituting the foreground reference image onto the combined image; and displaying a processed current image.

It is understood that both the foregoing general description and the following detailed description are exemplary and exemplary only, and are not restrictive of the invention as claimed.

### DESCRIPTION OF THE DRAWINGS

The accompanying drawings, which are incorporated in and constitute a part of the specification, illustrate preferred embodiments of the invention. Together with the description, they serve to explain the objects, advantages and principles of the invention. In the drawings:

FIG. 1A is a front perspective view of a prior art auto-darkening welding helmet;

FIG. 1B is a rear perspective view of the prior art auto-darkening welding helmet of FIG. 1A showing the interior of the helmet;

FIG. 2A is a front elevational view of a prior art auto-darkening welding helmet cartridge;

FIG. 2B is a rear elevational view of the prior art auto-darkening welding helmet cartridge of FIG. 2A;

FIG. 3A is a front perspective view of a mediated reality welding helmet according to the present invention;

FIG. 3B is a rear perspective view of a mediated reality welding helmet of FIG. 3A showing the interior of the helmet;

FIG. 4A is a front elevational view of a mediated reality welding helmet cartridge according to the present invention;

FIG. 4B is a rear elevational view of the mediated reality welding helmet cartridge of FIG. 4A;

FIG. 5 is a drawing of an exemplary weld bead used in mediated reality welding according to the present invention;

FIG. 6 is a block diagram of computer hardware used in the mediated reality welding helmet cartridge according to the present invention;

FIG. 7A is a flow chart of acts that occur to capture, process, and display mediated reality welding streaming video in a preferred embodiment of the present invention;

FIG. 7B is a flow chart continuing from and completing the flow chart of FIG. 7A;

FIG. 8 is a flow chart of acts that occur in the parallel processing of mediated reality welding streaming video in a preferred embodiment of the present invention;

FIG. 9 is a flow chart of acts that occur to composite mediated reality welding streaming video in a preferred embodiment of the present invention;

FIG. 10A is a picture of a background reference image used in compositing the mediated reality welding streaming video in a preferred embodiment of the present invention;

FIG. 10B is a picture of a first dark image used in compositing the mediated reality welding streaming video in a preferred embodiment of the present invention;

FIG. 10C is a picture of the first dark image composited with the background reference image in the mediated reality welding streaming video in a preferred embodiment of the present invention;

FIG. 10D is a picture of a last light torch and operator's hand in glove foreground reference image captured for subsequent use in processing the mediated reality welding streaming video in a preferred embodiment of the present invention;

FIG. 11A is a flow chart of acts that occur to generate a weld puddle vector for further processing by the mediated reality welding streaming video in a preferred embodiment of the present invention;

FIG. 11B is a flow chart continuing from and completing the flow chart of FIG. 11A;

FIG. 12A is a picture of a binary threshold applied to a weld puddle used in calculating a weld puddle vector for further processing by the mediated reality welding streaming video in a preferred embodiment of the present invention;

FIG. 12B is a picture of a weld puddle boundary and centroid used in calculating a weld puddle vector for further processing by the mediated reality welding streaming video in a preferred embodiment of the present invention;

FIG. 12C is a picture of an exemplary weld puddle vector used for further processing by the mediated reality welding streaming video in a preferred embodiment of the present invention;

FIG. 13A is a flow chart of acts that occur to extract the welding torch and operator's hand in glove for further processing by the mediated reality welding streaming video in a preferred embodiment of the present invention;

FIG. 13B is a flow chart continuing from and completing the flow chart of FIG. 13A;

FIG. 13C is a flow chart of the acts that occur to determine an initial vector of the torch and operator's hand in glove for further processing by the mediated reality welding streaming video in a preferred embodiment of the present invention;

FIG. 14A is a picture of a reference image of the welding torch and operator's hand in glove used for further processing by the mediated reality welding streaming video in a preferred embodiment of the present invention;

FIG. 14B is a picture of a binary threshold applied to the reference image of the welding torch and operator's hand in glove used for further processing by the mediated reality welding streaming video in a preferred embodiment of the present invention;

FIG. 14C is a picture of the extracted welding torch and operator's hand in glove used for further processing by the mediated reality welding streaming video in a preferred embodiment of the present invention;

FIG. 15A is a flow chart of acts that occur to construct mediated reality welding streaming video in a preferred embodiment of the present invention;

FIG. 15B is a flow chart continuing from and completing the flow chart of FIG. 15A; and

FIG. 16 is a picture of the generated mediated reality welding streaming video in a preferred embodiment of the present invention.

### DETAILED DESCRIPTION

The present invention is directed to a method and system for mediated reality welding. As discussed below, the method and system of the present invention uses mediated reality to improve operator or machine vision during welding operations.

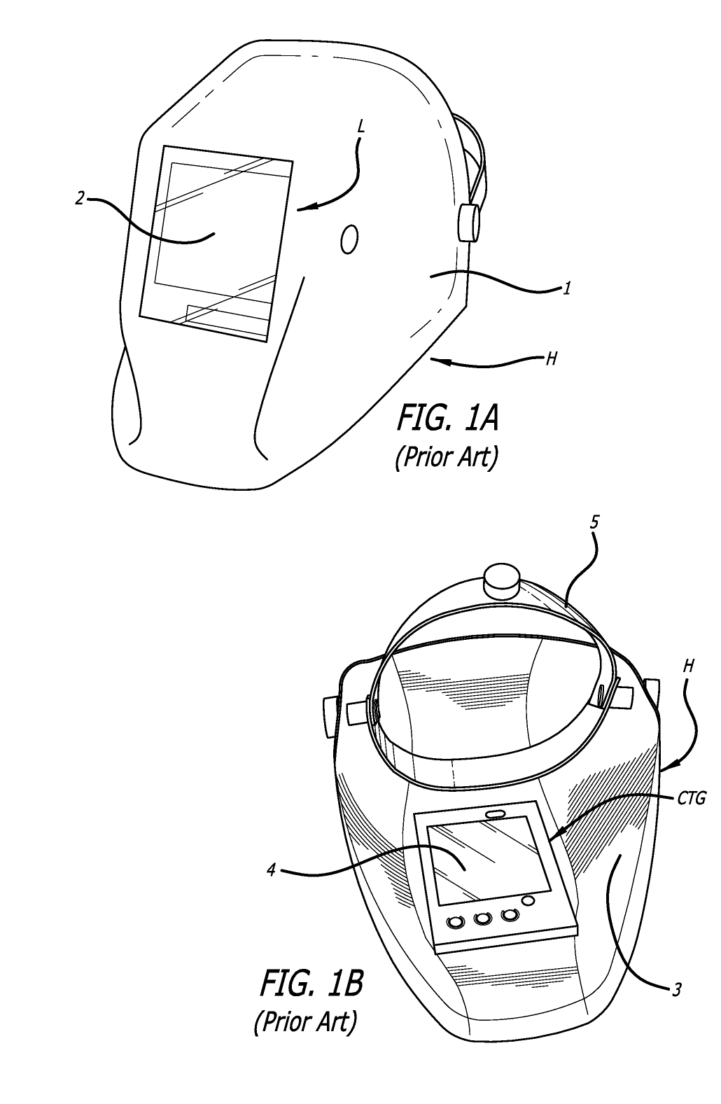

FIG. 1A depicts a prior art auto-darkening welding helmet H including a front mask 1 and a front 2 of a prior art battery powered auto-darkening cartridge CTG that protects an operator's face and eyes during welding.

FIG. 1B further depicts the prior art welding helmet H including an interior 3 of the welding helmet H, a back 4 of the prior art auto-darkening cartridge CTG, and an adjustable operator head strap 5 that allows for head size, tilt, and fore/aft adjustment which controls the distance between the operator's face and lens.

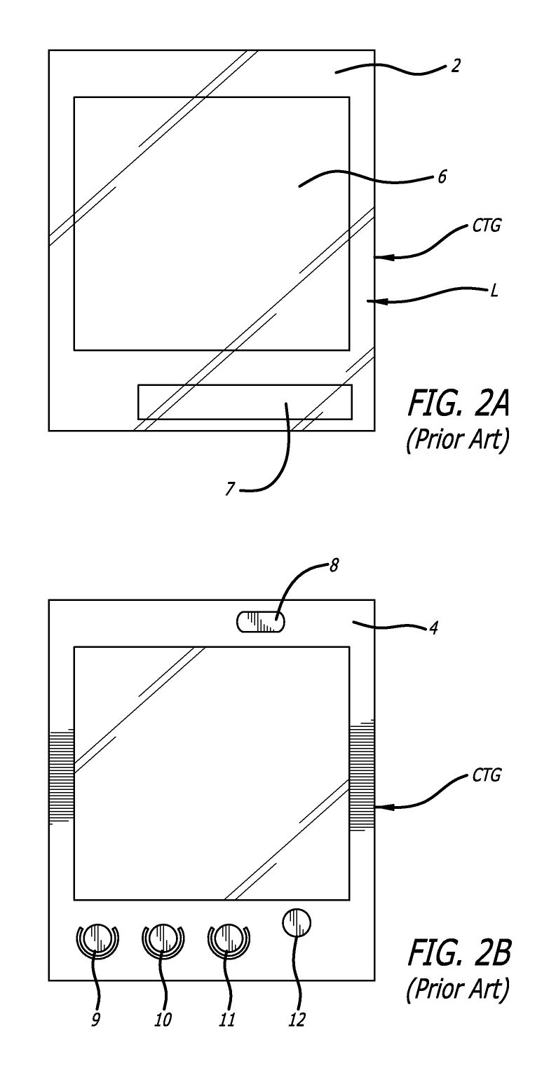

FIG. 2A depicts the front 2 of the prior art auto-darkening cartridge CTG. A protective clear lens L covers an auto-darkening filter 6 to protect the filter 6 from weld spatter and scratches. The prior art welding helmet H will automatically change from a light state (shade 3.5) to a dark state (shade 6-13) when welding starts. The prior art auto-darkening cartridge CTG contains sensors to detect the light from the welding arc, resulting in the lens darkening to a selected welding shade. The prior art auto-darkening cartridge CTG is powered by a replaceable battery (not shown) and solar power cell 7. The battery is typically located at the bottom corner of the cartridge.

FIG. 2B further depicts the back 4 of the prior art auto-darkening cartridge CTG. The controls of the prior art auto-darkening cartridge CTG include a shade range switch 8, a delay knob control 9 that is designed to protect the operator's eyes from the strong residual rays after welding, a sensitivity knob 10 that adjusts the light sensitivity when the helmet is used in the presence of excess ambient light, a shade dial 11 to set the desired shade, and a test button 12 to preview shade selection before welding. The industry standard auto-darkening cartridge size is 4.5 inches wide by 5.25 inches high.

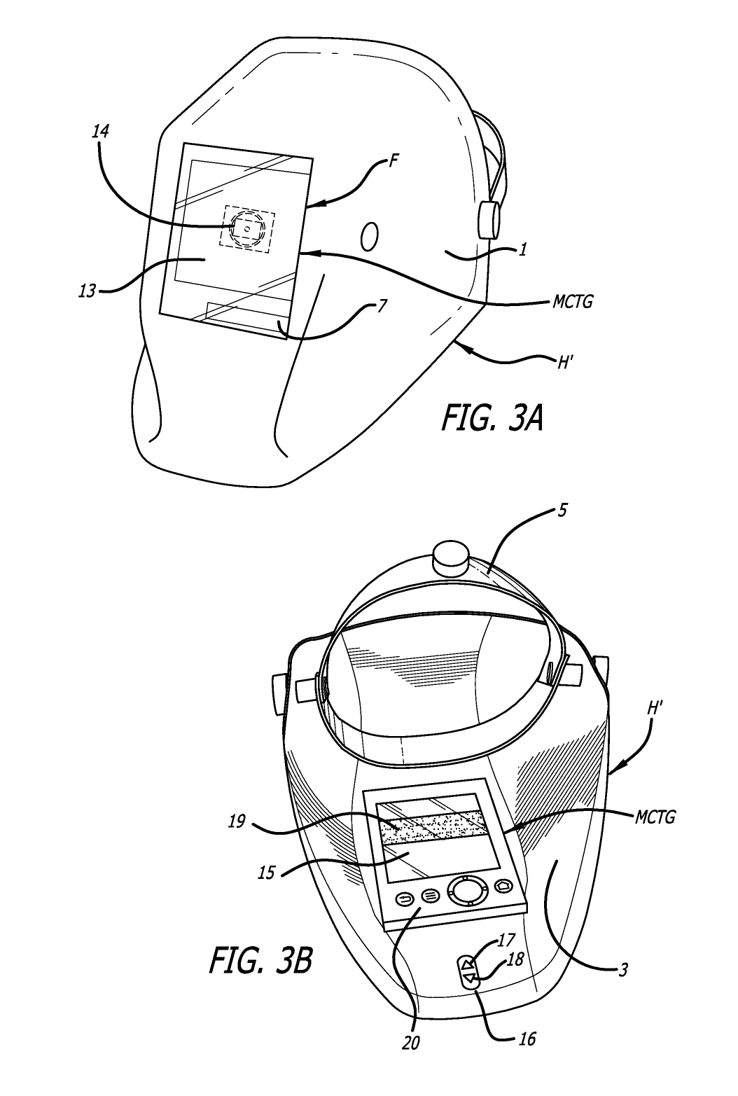

FIG. 3A shows a modified welding helmet H′. The modified welding helmet H′ includes many of the features of the prior art welding helmet H, but has been modified to accommodate use of a mediated reality welding cartridge MCTG.

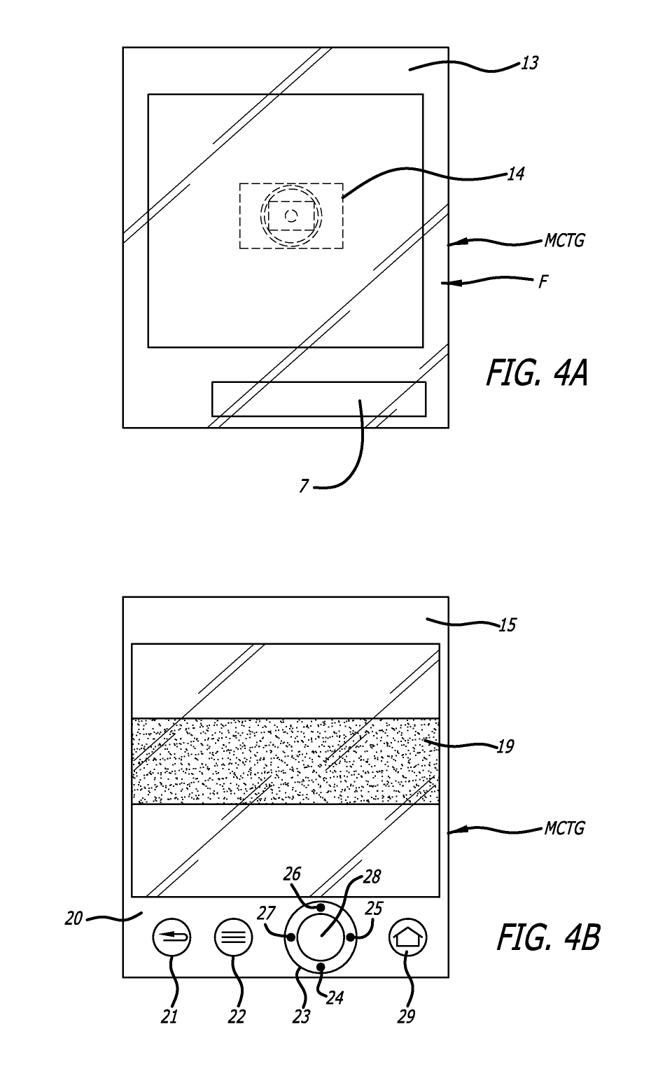

The modified helmet H′ includes the front mask 1 that has been modified to accept the mediated reality welding cartridge MCTG. In FIGS. 3A and 4A, a front 13 of the mediated reality welding cartridge MCTG is shown with a camera (or image sensor) 14 behind a clear protective cover and auto-darkening filter F that protects the operator's face and eyes during welding. The mediated reality welding cartridge MCTG cartridge is powered by a replaceable battery (not shown) and solar power cell 7. The battery is typically located at the bottom corner of the cartridge.

FIG. 3B further shows the interior 3 of the modified welding helmet H′ that has been modified to accept the mediated reality welding cartridge MCTG. As shown in FIGS. 3B and 4B, a back 15 of the mediated reality welding cartridge MCTG includes a display screen 19 and an operator focus control 16 to focus the camera (or image sensor) 14 for operator viewing of the work piece being welded displayed on the display screen 19 using a zoom in button 17 or a zoom out button 18. The back 15 of the mediated reality welding cartridge MCTG also includes operator controls 20 for accessing cartridge setup including shade adjustment, delay, sensitivity, and test. The mediated reality welding cartridge MCTG is programmed with mediated reality welding application software, and the operator control 20 is also used for accessing the mediated reality welding application software. The operator control 20 has tactile feedback buttons including: “go back” button 21; “menu” button 22; a mouse 23 containing “up” button 26, “down” button 24, “right” button 25, “left” button 27, and “select” 28 button; and a “home” 29 button.

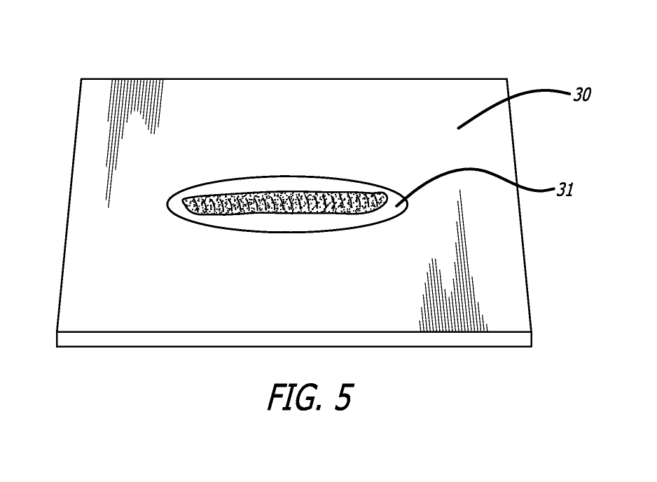

FIG. 5 shows an exemplary piece of steel 30 with a weld bead 31 which will be used to illustrate mediated reality welding in a preferred embodiment of the present invention.

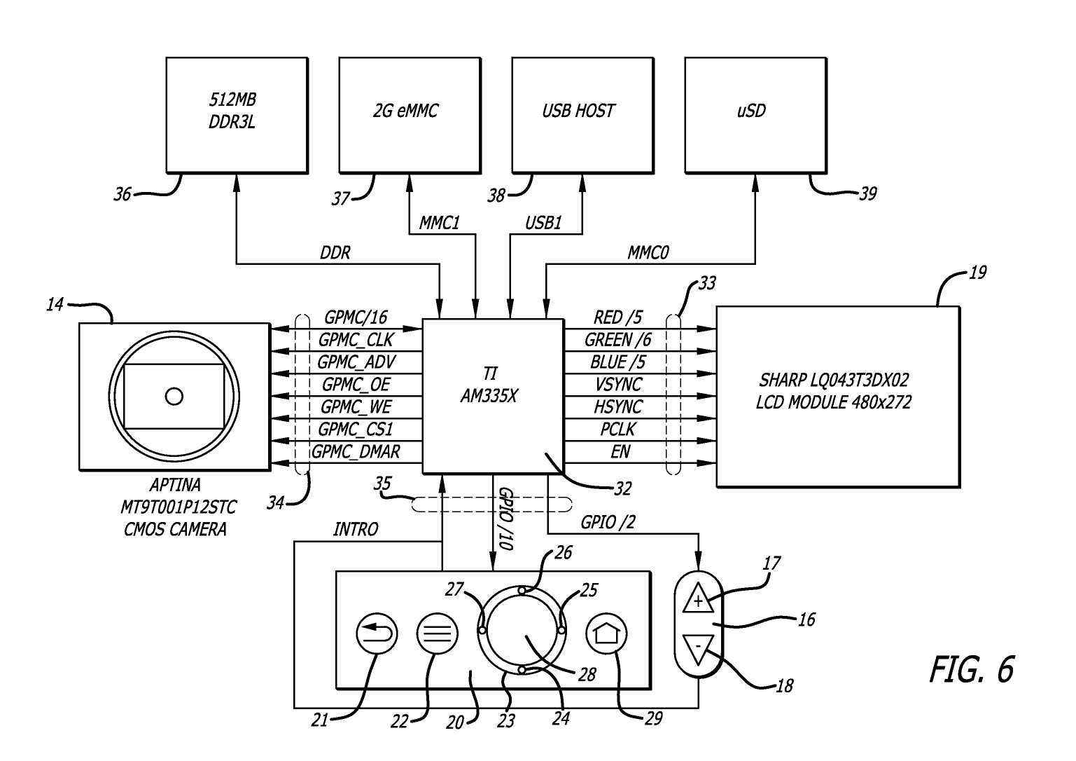

FIG. 6 is a block diagram of the computer hardware used in the mediated reality welding cartridge MCTG. The hardware and software of the cartridge captures, processes, and displays real-time streaming video, and provides operator setup and mediated reality welding application software. A microprocessor 32 from the Texas Instruments AM335x Sitara microprocessor family can be used in a preferred embodiment. The AM335x is based on the ARM (Advanced Risc Machines) Cortex-A8 processor and is enhanced with image, graphics processing, and peripherals. The operating system used in the computer hardware of a preferred embodiment is an embedded Linux variant.

The AM335x has the necessary built-in functionality to interface to compatible TFT (Thin Film Transistor) LCD (Liquid Crystal Display) controllers or displays. The display screen 19 can be a Sharp LQ043T3DX02 LCD Module capable of displaying 480 by 272 RGB (Red, Green, Blue) pixels in WQVGA (Wide Quarter Video Graphics Array) resolution. The display screen 19 is connected to the AM335x, and receives signals 33 from the AM335x that support driving an LCD display. The AM335x, for example, outputs signals 33 including raw RGB data (Red/5, Green/6, Blue/5) and control signals Vertical Sync (VSYNC), Horizontal Sync (HSYNC), Pixel Clock (PCLK) and Enable (EN).

Furthermore, the AM335x also has the necessary built-in functionality to interface with the camera (or image sensor) 14, and the camera (or image sensor) 14 can be a CMOS Digital Image Sensor. The Aptina Imaging MT9T001P12STC CMOS Digital Image Sensor 14 used in a preferred embodiment is a 3-Megapixel sensor capable of HD (High Definition) video capture. The camera (or image sensor 14) can be programmed for frame size, exposure, gain setting, electronic panning (zoom in, zoom out), and other parameters. The camera (or image sensor) 14 uses general-purpose memory controller (GPMC) features 34 of the AM335x (microprocessor 32) to perform a DMA (Direct Memory Access) transfer of captured video to memory 36 in the exemplary form of 512MB DDR3L (DDR3 Low-Voltage) DRAM (Dynamic Random-Access Memory) 36. DDR3, or double data rate type three synchronous dynamic random-access memory, is a modern type of DRAM with a high bandwidth interface. The AM335x provides a 16 bit multiplexed bidirectional address and data bus (GPMC/16) for transferring streaming camera video data to the 512MB DDR3L DRAM and GPMC control signals including Clock (GPMC_CLK), Address Valid/Address Latch Enable (GPMC_ADV), Output Enable/Read Enable (GPMC_OE), Write Enable (GPMC_WE), Chip Select (GPMC_CS1), and DMA Request (GPMC_DMAR).

The tactile feedback buttons of the operator control 20 and the operator focus control 16 are scanned for a button press by twelve General Purpose Input/Output lines, GPIO/10 and GPIO/2. If a button is pressed, an interrupt signal (INTR0) 35 signals the microprocessor 32 to determine which button was pressed.

The embedded Linux operating system, boot loader, and file system along with the mediated reality application software is stored in memory 37 in the exemplary form of the 2 Gigabyte eMMC (embedded MultiMediaCard) memory. The memory 37 is a non-transitory computer-readable medium facilitating storage and execution of the mediated reality application software. A Universal Serial Bus (USB) host controller 38 is provided for communication with a host system such as a laptop personal computer for diagnostics, maintenance, feature enhancements, and firmware upgrades. Furthermore, a micro Secure Digital (uSD) card interface 39 is integrated into the cartridge and provides removable non-volatile storage for recording mediated reality welding video, feature, and firmware upgrades.

Real-time streaming video applications are computationally demanding. As discussed above, a preferred embodiment of the present invention relies on the use of an ARM processor for the microprocessor 32. However, alternate preferred embodiments may use a single or multiple core Digital Signal Processors (DSP) in conjunction with an ARM processor to offload computationally intensive image processing operations. A Digital Signal Processor is a specialized microprocessor with its architecture optimized for the operational needs of signal processing applications. Digital signal processing algorithms typically require a large number of mathematical operations to be performed quickly and repeatedly on a series of data samples. Signals (perhaps from audio or video sensors) are constantly converted from analog to digital, manipulated digitally, and then converted back to analog form. Many DSP applications have constraints on latency; that is, for the system to work, the DSP operation must be completed within some fixed time, and deferred (or batch) processing is not viable. The Texas Instruments C667x DSP family is an example of the type and kind of DSP that could be used in an alternate preferred embodiment.

In addition to ARM processors and DSPs, Accelerator system-on-chip (SoC) could be used within the framework of the preferred embodiment to provide an alternate preferred embodiment. Examples of dedicated accelerator system-on-chip modules include specific codec's coder-decoder. A codec is a device or software capable of encoding or decoding a digital data stream or signal. A codec encodes a data stream or signal for transmission, storage, or encryption, or decodes it for playback or editing. Codecs are used in videoconferencing, streaming media, and video editing applications. The computer hardware used for the mediated reality cartridge could include any combination of ARM, DSP, and SoC hardware components depending upon performance and feature requirements. Furthermore, different types of cameras and displays including but not limited to heads up displays, etc., could be used in preferred embodiments.

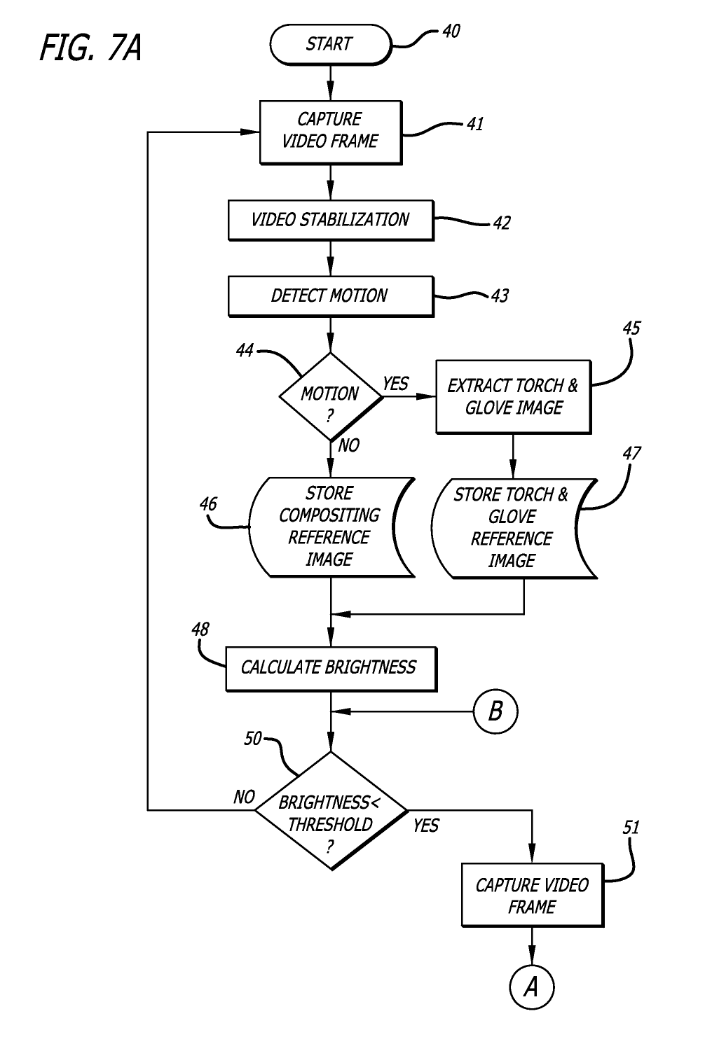

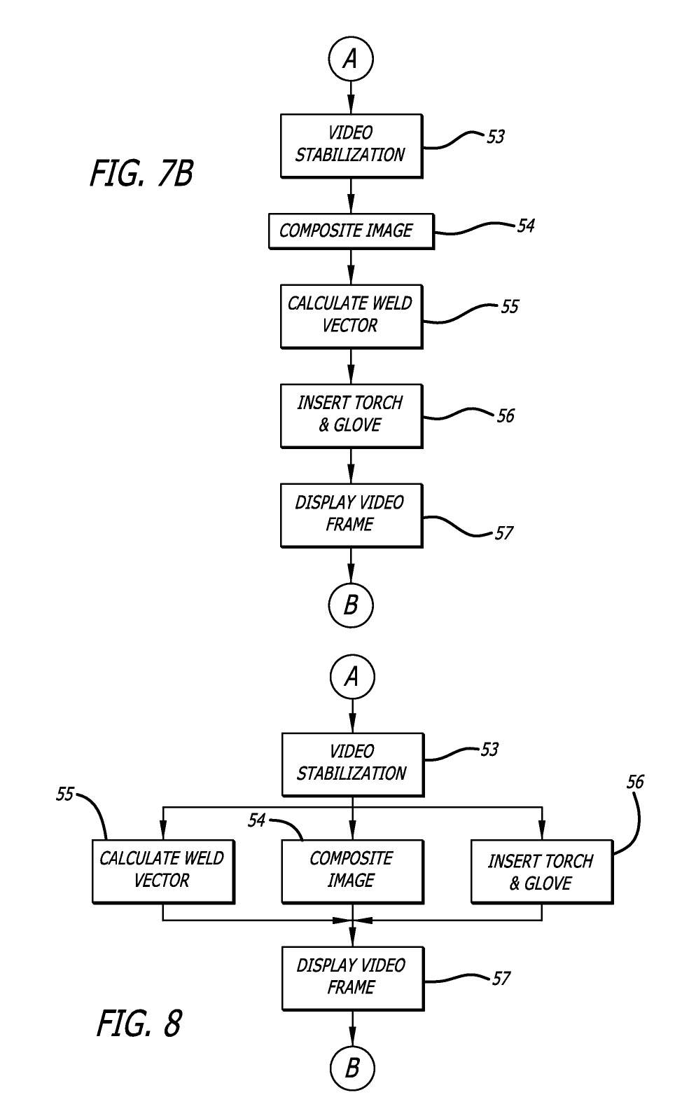

FIGS. 7A and 7B are directed to a flow chart of acts that occur to capture, process, and display mediated reality welding streaming video in a preferred embodiment. The processing starts at block 40 after system initialization, the booting of the embedded Linux operating system, and the loading of the mediated reality welding application software. One or more video frames are captured by camera (or image sensor) 14 and stored in memory 36 at block 41. To adjust for operator head movement, a video stabilization algorithm is used at block 42. The video stability algorithm uses block matching or optical flow to process the frames in memory 36 and the result is stored therein.

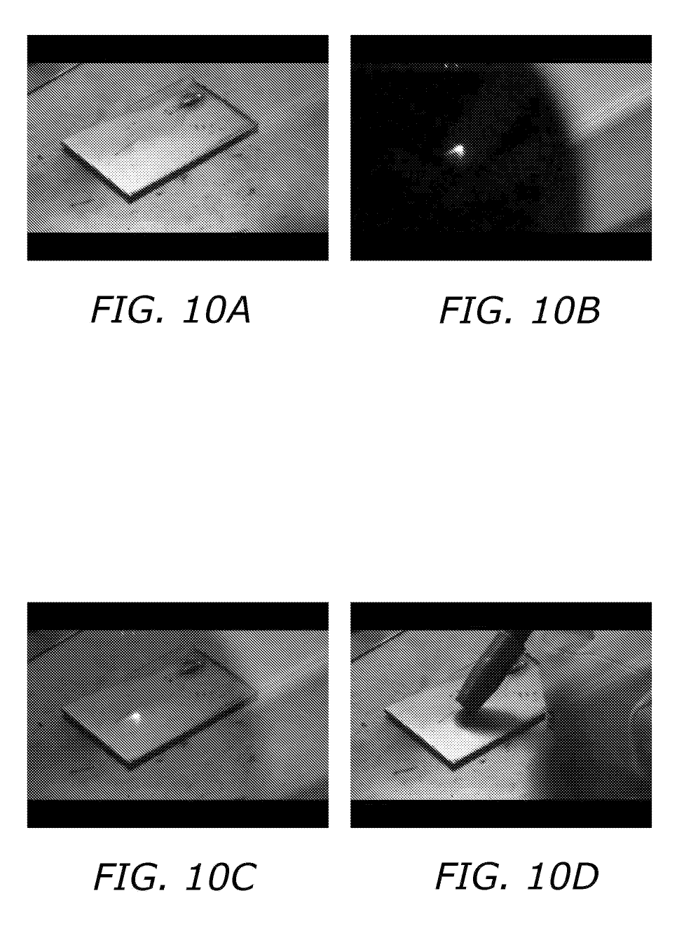

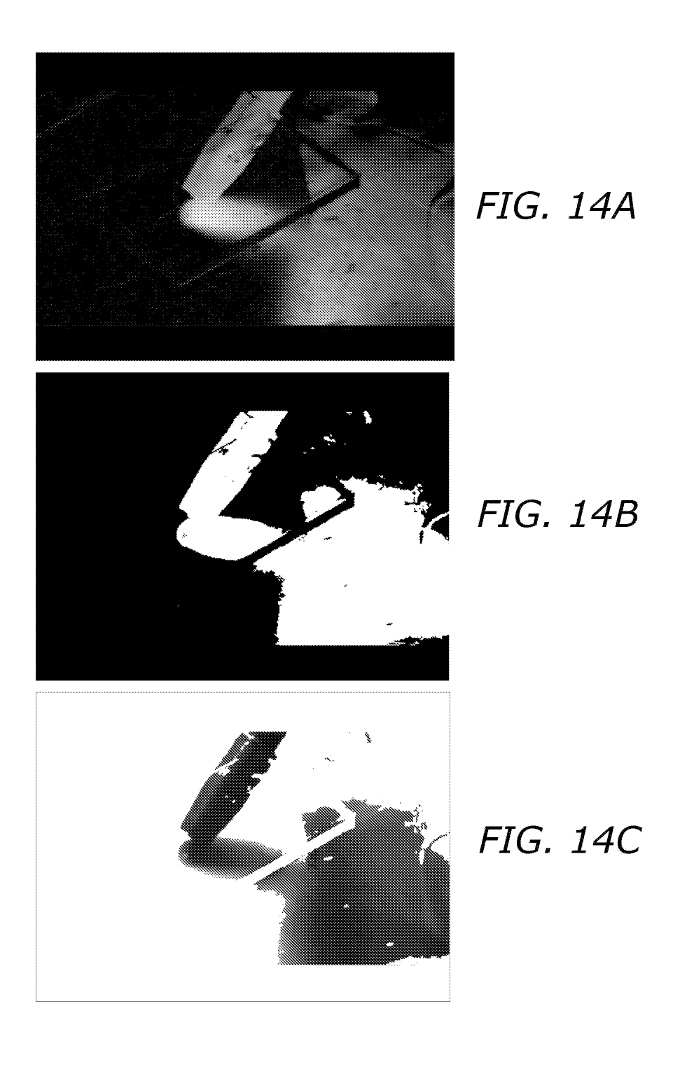

A simple motion detection algorithm is used at block 43 to determine if the operator's welding torch and glove appear in the frame (FIG. 10D). If at block 44 it is determined that the torch and glove appear in the frame, the process continues from block 44 to block 45 where an algorithm to extract the RGB torch and glove foreground image from the background image of the material being welded is executed. The extracted RGB torch and glove reference image (FIG. 14C) is stored in a buffer at block 47 for further processing. If block 44 it is determined that a torch and glove image is not detected (i.e., the torch and glove do not appear in the frame), the process continues from block 44 to block 46 where the current image is stored in a buffer as a RGB reference image (FIG. 10A) for use in the compositing algorithm at block 54.

Brightness is calculated at block 48. The brightness calculation is used to determine when the welding arc causes the helmet shade to transition from light to dark (FIGS. 10A and 10B). If at block 50 it is determined that the brightness is less than the threshold, blocks 41-50 are repeated. Otherwise, if at block 50 it is determined that the brightness is greater than the threshold, the video frame capture continues at block 51.

Instead of using a brightness calculation in software at block 48 to execute blocks 51-57, a hardware interrupt could be used when the welding helmet shade transitions from light to dark. The welding helmet auto-darkening filter has an existing optical sensing circuit that detects the transition from light to dark and could provide an interrupt that runs an interrupt routine executing blocks 51-57.

As was discussed, if at block 50 it is determined that the brightness is greater than the threshold, video frame capture continues at block 51. One or more video frames are captured by camera (or image sensor) 14 and stored in memory 36 at block 51. To adjust for operator head movement, a video stabilization algorithm (such as block matching or optical flow) is used at block 53 to process the frames in memory 36 and the result is stored therein.

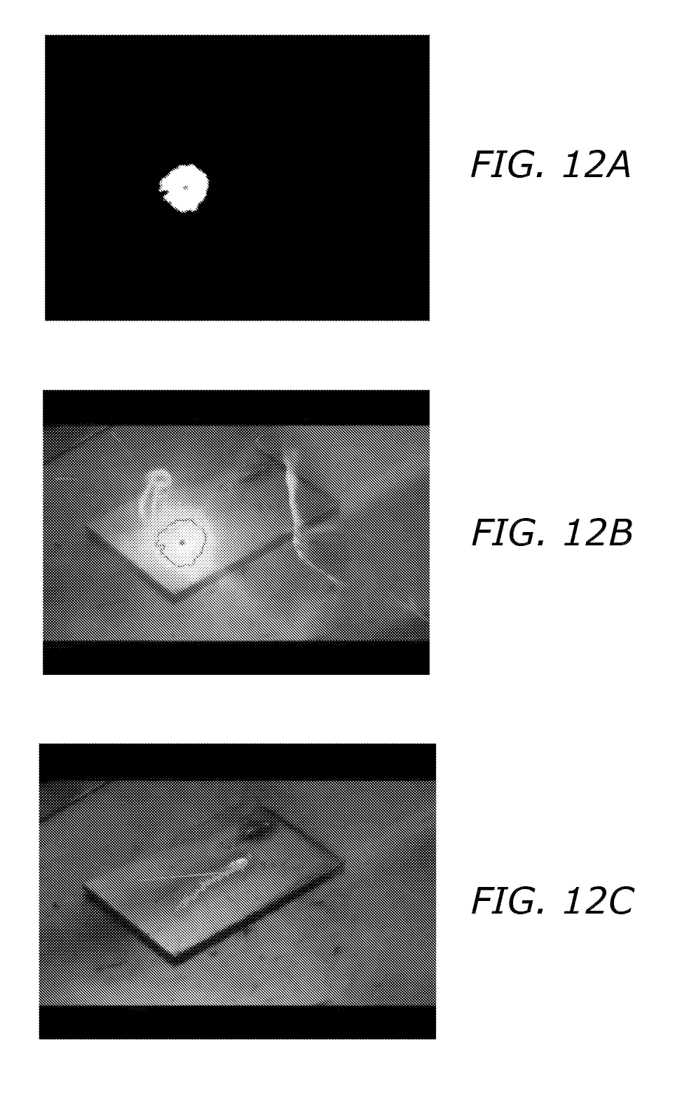

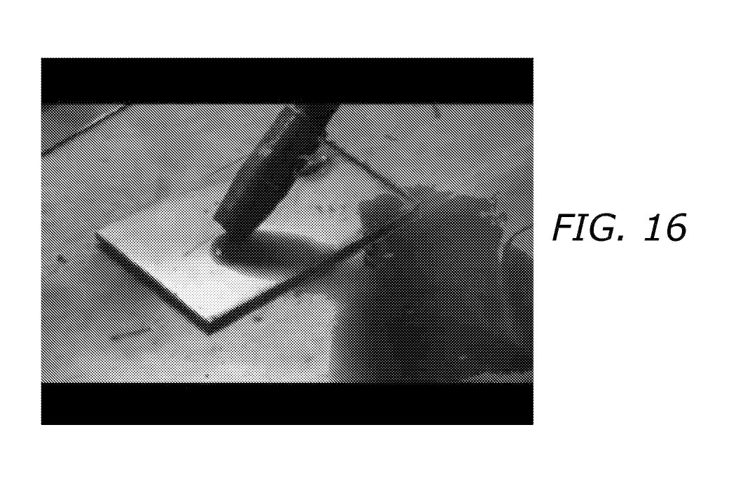

The currently captured RGB frame (FIG. 10B) is composited with the RGB composite reference image (FIG. 10A) at block 54. The process of compositing allows two images to be blended together. In the case of mediated reality welding, a RGB reference image is used for compositing. This reference image is the last known light image (FIG. 10A) without the torch and glove captured by the camera (or image sensor) 14 before the welding arc darkens the shade. Once the shade is darkened, the camera (or image sensor) 14 captures the dark images (FIG. 10B) frame by frame and composites the dark images with the light reference image. The result is that the dark images are now displayed to the operator on the display screen 19 as pre-welding arc light images which greatly improve operator visibility during a welding operation. At this point, the light image (FIG. 10C) lacks the torch and glove (FIG. 10D). By using a binary mask (FIG. 12A) on the weld puddle of the current dark image (FIG. 10B), a centroid for the weld puddle (FIG. 12B) can be used to calculate a vector (wx, wy) at block 55 that will provide a location where the center of the torch tip from the extracted torch and glove reference image (FIGS. 14B, 14C) can be added back into the current composited image (FIG. 10C) at block 56. The resulting image (FIG. 16) is displayed at block 57 to the operator on the display screen 19 and the process repeats starting at block 50.

Real-time streaming video applications are computationally intensive. FIG. 8 illustrates an alternate preferred embodiment of FIGS. 7A and 7B by performing the acts of weld vector calculation 55, image compositing 54, and torch and glove insertion 56 in parallel to facilitate display of the resulting image at block 57. This could be accomplished in software using multiple independent processes which are preemptively scheduled by the operating system, or could be implemented in hardware using either single or multiple core ARM processors or offloading image processing operations onto a dedicated single or multiple core DSP. A combination of software and dedicated hardware could also be used. Whenever possible, parallel processing of the real-time video stream will increase system performance and reduce latency on the display screen 19 potentially experienced by the operator during the welding operation. Furthermore, any pre-processing operations involving reference images are also desirable to reduce latency on the display screen 19.

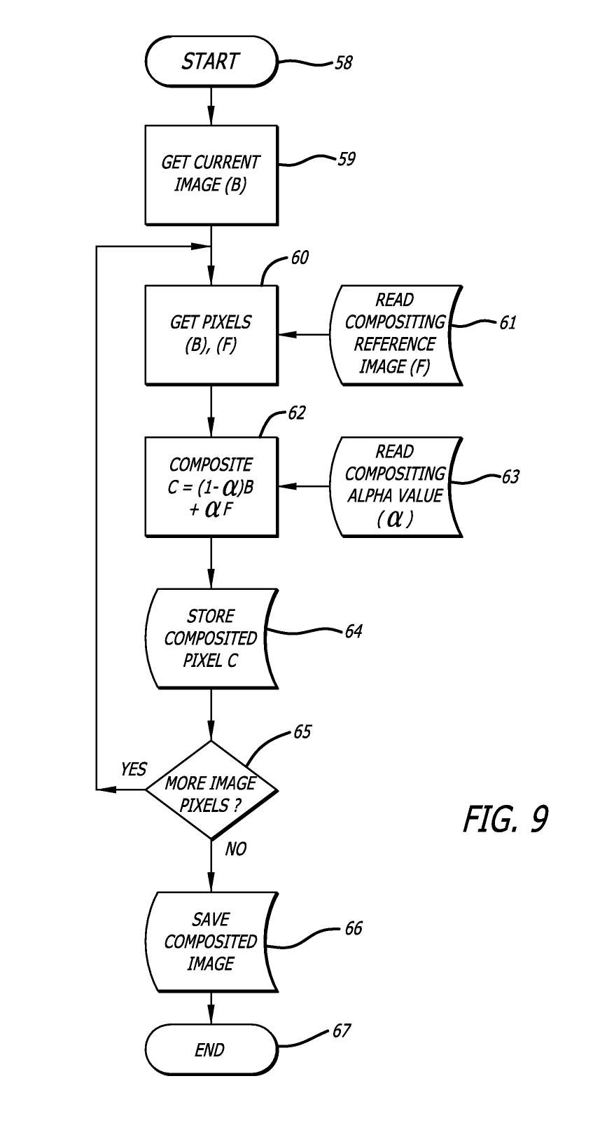

The detailed acts to composite the current dark image (FIG. 10B) with the last light reference image (FIG. 10A) before the introduction of the welding torch and glove (FIG. 10D) in a video frame are shown in FIG. 9. Compositing is the combining of visual elements from separate sources into single images, often to create the illusion that all those elements are parts of the same scene. The video frames captured by camera (or image sensor) 14 can be categorized as “light” frames and “dark” frames. A “light” frame is an image as is seen by the welding helmet auto-darkening filter before the torch is triggered by the operator to begin the welding operation. A “dark” frame is the image as is seen by the auto-darkening filter after the torch is triggered by the operator to begin the welding operation. A reference background image (FIG. 10A) is chosen for compositing the “dark” frames (FIG. 10B) to make them appear as “light” frames during the welding operation to greatly improve the visual environment for the operator. Each “dark” frame is composited with the reference image (FIG. 10C) and saved for further processing.

The specific reference image chosen is the last light frame available (FIG. 10A) before the welding torch and operator's glove start to show up in the next frame. A buffer of frames stored at blocks 46 and 47 is examined to detect the presence of the torch and glove so real-time selection of reference images can be accomplished. Once the torch is triggered, the saved compositing reference image (FIG. 10A) and torch and glove reference image (FIG. 10D) is used in real-time streaming video processing. An interrupt driven approach where an interrupt is generated by the auto-darkening sensor on the transition from “light” to “dark” could call an interrupt handler which would save off the last “light” image containing the torch and glove.

In FIG. 9, the compositing process starts at block 58, and the current dark image B (FIG. 10B) is obtained at block 59. Block 60 begins by reading each RGB pixel in both the current image B (FIG. 10B), and the reference image F (FIG. 10A) from block 61. Block 62 performs compositing on a pixel-by-pixel basis using a compositing alpha value α (stored in memory at block 63) and the equation C=(1−α)B+αF. The composited pixel C is stored in memory at block 64. If at block 65 it is determined that more pixels need to be processed in the current RGB image, the process continues at block 60; otherwise, the composited image (FIG. 10C) is saved into memory at block 66 for further processing. The compositing process of FIG. 9 ends at 67 until the next frame needs to be composited.

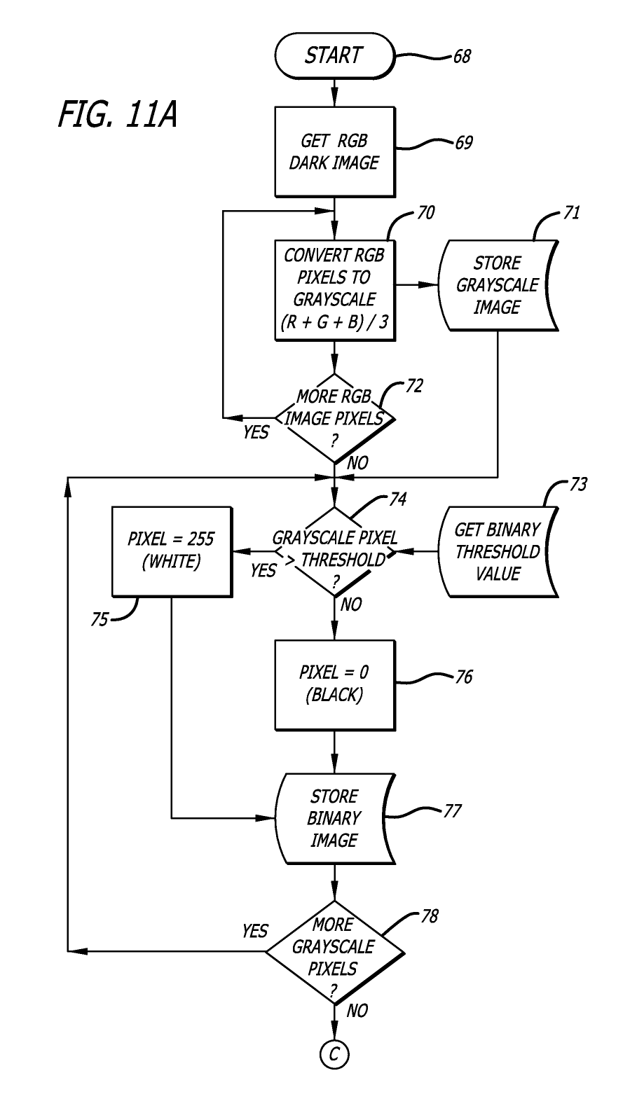

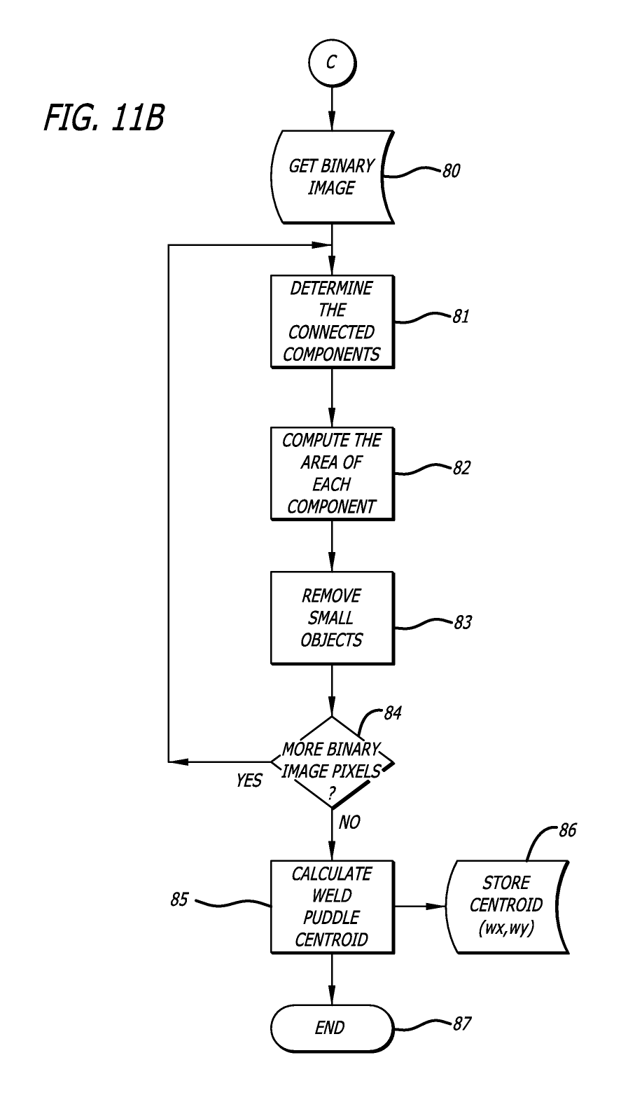

FIGS. 11A, 11B, and 11C disclose a flow chart of the acts that occur to generate a weld puddle vector for further processing by the mediated reality welding streaming video in a preferred embodiment. While the composited video dramatically enhances the luminosity of the visual welding experience for the operator, details such as the welding torch and glove are primarily absent. Also, the weld puddle itself is the brightest part of the image, just as it was before. Since the last “light” torch and glove image (FIG. 10D) is the only “light” image remaining that can be used to add back into the composited video, the torch and glove need to be extracted from this image and moved along with the weld puddle. The bright weld puddle can be used advantageously by using a binary threshold on each frame to isolate the weld puddle, then measuring the mathematical properties of the resulting image region, and then calculating a centroid to determine the x and y coordinates of the weld puddle center.

A centroid is a vector that specifies the geometric center of mass of the region. Note that the first element of centroid is the horizontal coordinate (or x-coordinate) of the center of mass, and the second element is the vertical coordinate (or y-coordinate) of the center of mass. All other elements of a centroid are in order of dimension. A centroid is calculated for each frame and used to construct a x-y vector of the weld puddle movement. This vector will subsequently be used to add back in the torch and glove image on the moving image to allow the torch and glove to move along with the weld puddle. The results of this operation are shown in FIGS. 12A, 12B and 12C.

Also, by measuring the weld puddle area, it is possible to improve feedback to the operator regarding weld quality. Useful information displayed to the operator may also include 1) weld speed, 2) weld penetration, 3) weld temperature, and 4) distance from torch tip to material. All of these aforementioned factors have a great impact on weld quality.

Calculation of the weld puddle vector starts in FIG. 11A at block 68. The current RGB dark image (FIG. 10B) is read from memory 36 at block 69, and the RGB dark image (FIG. 10B) is converted to a grayscale image at block 70. The image is converted to grayscale in order to allow faster processing by the algorithm. When converting a RGB image to grayscale, the RGB values are taken for each pixel and a single value is created reflecting the brightness of that pixel. One such approach is to take the average of the contribution from each channel: (R+G+B)/3. However, since the perceived brightness is often dominated by the green component, a different, more “human-oriented”, method is to take a weighted average, 0.3R+0.59G+0.11B. Since the image is going to be converted to binary (i.e., each pixel will be either black or white), the formula (R+G+B)/3 can be used. As each RGB pixel is converted to a grayscale pixel at block 70, the grayscale image stored into memory 36 at block 71. If at block 72 it is determined that there are more pixels in the RGB image to be converted 72, processing continues at block 70; otherwise, the RGB to grayscale conversion has been completed.

After the conversion to grayscale, the image needs to next be converted from grayscale to binary starting at block 74. Converting the image to binary is often used in order to find a ROI (Region of Interest), which is a portion of the image that is of interest for further processing. The intention is binary, “Yes, this pixel is of interest” or “No, this pixel is not of interest”. This transformation is useful in detecting blobs and reduces the computational complexity. Each grayscale pixel value (0 to 255) is compared at block 74 to a threshold value from block 73 contained in memory. If at block 74 it is determined that the grayscale pixel value is greater than the threshold value, the current pixel is set to 0 (black) at block 76; otherwise, the current pixel is set to 255 (white) at block 75. The result of the conversion is stored pixel by pixel at block 77 until all of the grayscale pixels have been converted to binary pixels at block 78.

Next mathematical operations are performed on the resulting binary image at block 80. Once region boundaries have been detected by converting the image to binary, it is useful to measure regions which are not separated by a boundary. Any set of pixels which is not separated by a boundary is called connected. Each maximal region of connected pixels is called a connected component with the set of connected components partitioning an image into segments. The case of determining connected components at block 81 in the resulting binary image can be straight forward, since the weld puddle typically produces the largest connected component. Detection can be accomplished by measuring the area of each connected component at block 82. However, in order to speed up processing, the algorithm uses a threshold value to either further measure or ignore components that have a certain number of pixels in them. The operation then quickly identifies the weld puddle in the binary image by removing the smaller objects from the binary image at block 83. The process continues until all pixels in the binary image have been inspected at block 84. At this point, a centroid is calculated at block 85 for the weld puddle. A centroid is the geometric center of a two-dimensional region by calculating the arithmetic mean position of all the points in the shape. FIG. 12A shows the binary image, the resulting region of the detected weld puddle and the centroid in the middle of the weld puddle. FIG. 12B illustrates the area of the weld puddle and corresponding centroid overlaid on the image that was processed. The current weld puddle centroid (wx, wy) is stored into memory 36 at block 86 for further processing and the calculation algorithms have completed at block 87 until the next image is processed. For illustrative purposes, FIG. 12C plots the weld vectors for a simple welding operation shown in FIG. 5. In the real-time streaming video application of the preferred embodiment, each vector calculation is used on its own as it occurs in subsequent processing acts.

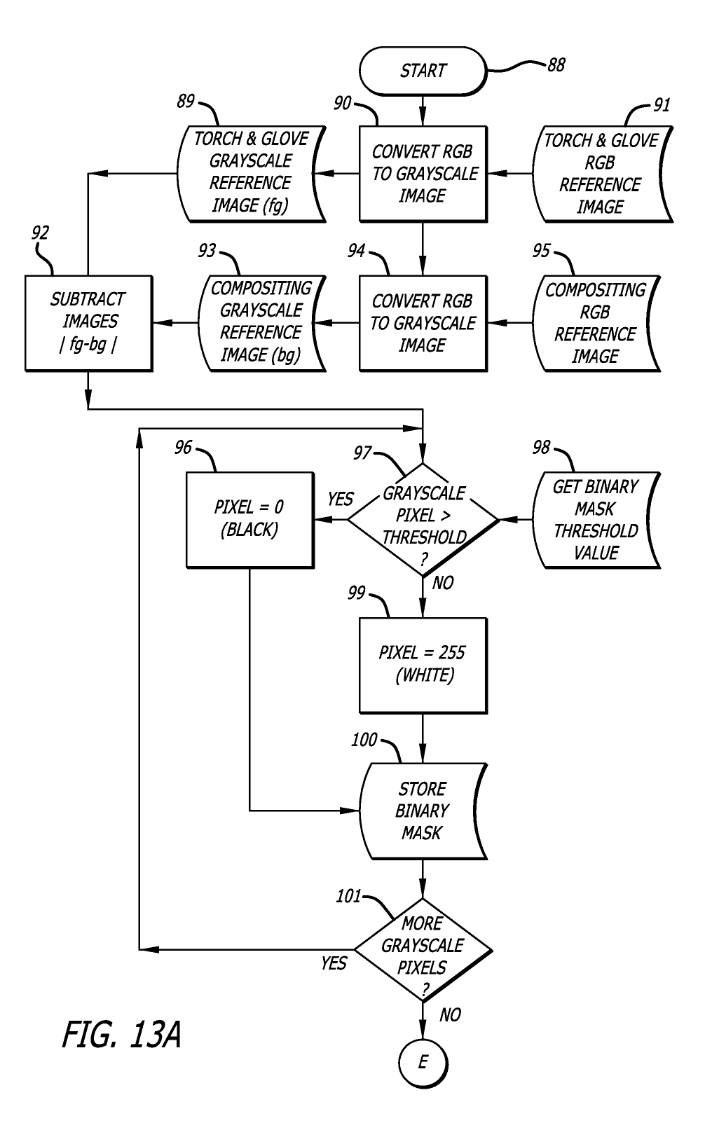

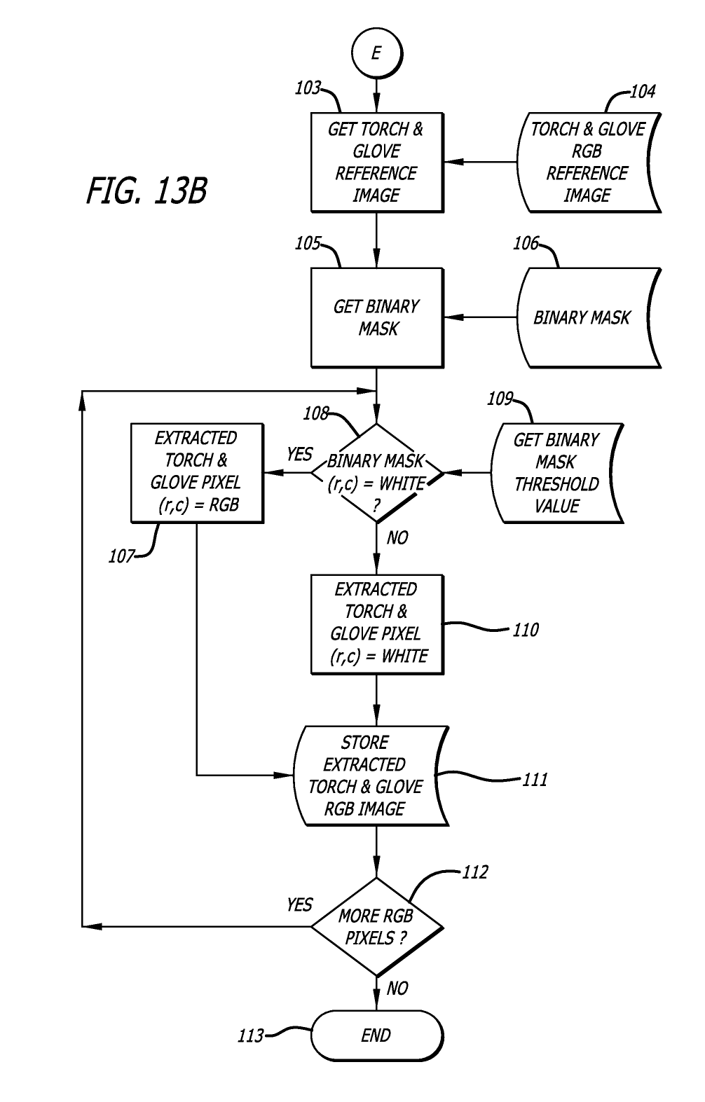

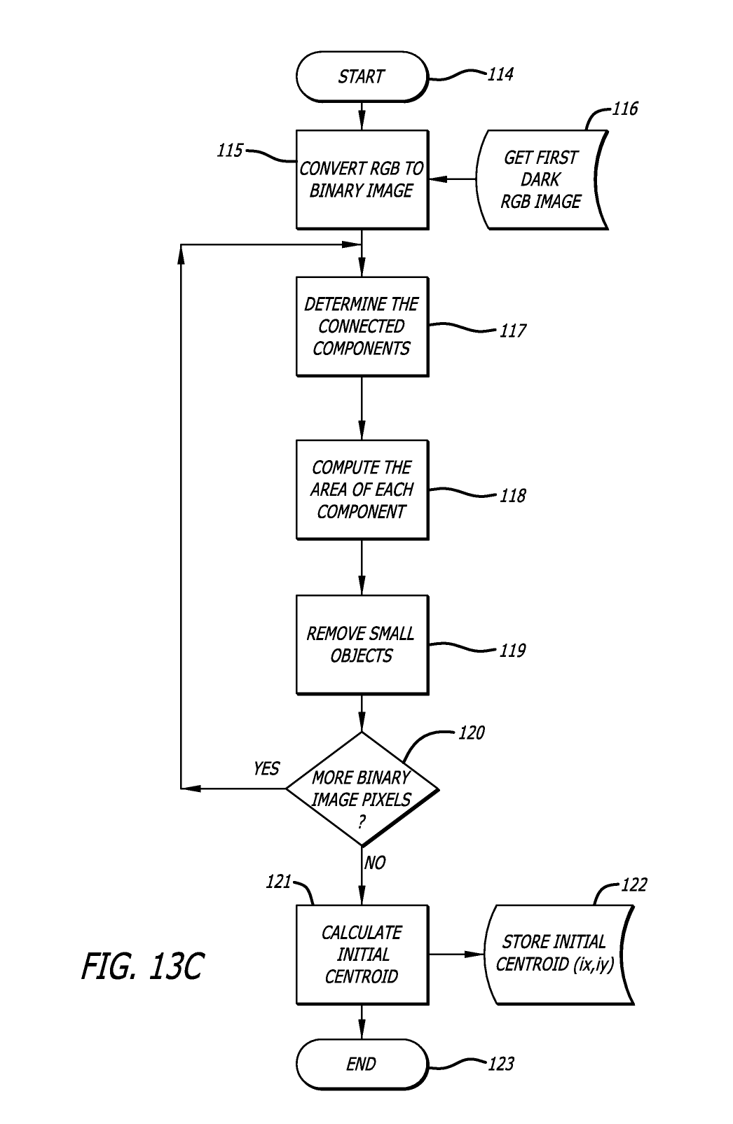

FIGS. 13A, 13B, and 13C extract the welding torch and glove from the last “light” torch and glove image. FIG. 10D is the only “light” image remaining that can be used to add back into the composited video. The welding torch and glove are extracted using the following process: 1) subtract the foreground image minus the background image using i) the last background reference image (FIG. 10A) before the torch and glove (FIG. 10D) is introduced into the next frame, and then ii) subtract the last “light” torch and glove image (FIG. 14A); 2) binary threshold the subtracted image to produce a mask for the extraction of the torch and glove (FIG. 14B); and 3) extract the RGB torch and glove image. The results are shown in FIG. 14C. A centroid is calculated for the resulting image. This initial centroid (ix,iy) will be used in the calculations required to take the torch and glove and move it along the weld puddle vector (wx,wy) to create the mediated reality welding streaming video (FIG. 16).

Starting in FIG. 13A at block 88, the RGB torch and glove reference image (FIG. 10D) is read from memory 36 at block 91, and the RGB torch and glove reference image (FIG. 10D) is converted to a grayscale image as was previously discussed at block 90. The result is stored back into memory 36 at block 89 as a foreground (fg) image. The compositing RGB reference image (FIG. 10A) is read from memory 36 at block 95, converted to a grayscale image at block 94, and stored back into memory 36 at block 93. The absolute value of the foreground (fg) image minus the background (bg) image is calculated at block 92 (FIG. 14A) extracting the torch and glove for further processing at block 97. The extracted image is converted to a binary image (FIG. 14B) by reading a threshold value from memory 36 at block 98 and comparing the pixels in the grayscale image at block 97. If the grayscale pixel is greater than the threshold, the pixel is set to white at block 99 otherwise the pixel is set to black at block 96. The result is stored pixel by pixel as a binary mask at block 100 until all of the grayscale pixels are converted to binary pixels at block 101. If the conversion is done, processing continues to FIG. 13B; otherwise, processing continues at block 97.

Next in FIG. 13B, the torch and glove RGB reference image (FIG. 10D) from block 104 is read from memory 36 and obtained by block 103, and the torch and glove binary mask (FIG. 14B) from block 106 is read from memory 36 and obtained by block 105. In order to extract the RGB torch and glove, a binary mask is read from memory 36 and obtained by block 109. Next, the extracted RGB torch and glove is placed on a white background starting at block 108 where each RGB and mask pixel by row and column (r,c) is processed. If at block 108 it is determined the current pixel in the binary mask is white, the corresponding pixel from the RGB image is placed in the extracted image at block 107; otherwise, the pixel in the RGB image is set to white at block 110. Each processed pixel is then stored at block 111, and, if at block 112 it is determined that there are more pixels in the RGB image, processing continues at block 108; otherwise, no more pixels are needing to be processed at the algorithm ends at block 113. The result of the algorithm of FIGS. 13A and 13B produces an extracted torch and glove RGB image FIG. 14C.

The final act in preparing the extracted image for subsequent use is to calculate the location of the welding torch's tip using a centroid. The algorithm of FIG. 13C is performed once to determine the centroid. In FIG. 13C, acts 114-121 are similar to acts 80-85 of FIG. 11B which have previously been discussed. The initial centroid (ix,iy) of the extracted torch and glove image is stored at block 122 and processing ends at block 123. For illustrative purposes, the centroid is overlaid on FIGS. 14A-14C. It will be appreciated by one of ordinary skill in the art that techniques such as video inpainting, texture synthesis or matting, etc., could be used in the preceding algorithm (FIGS. 13A, 13B) to accomplish the same result.

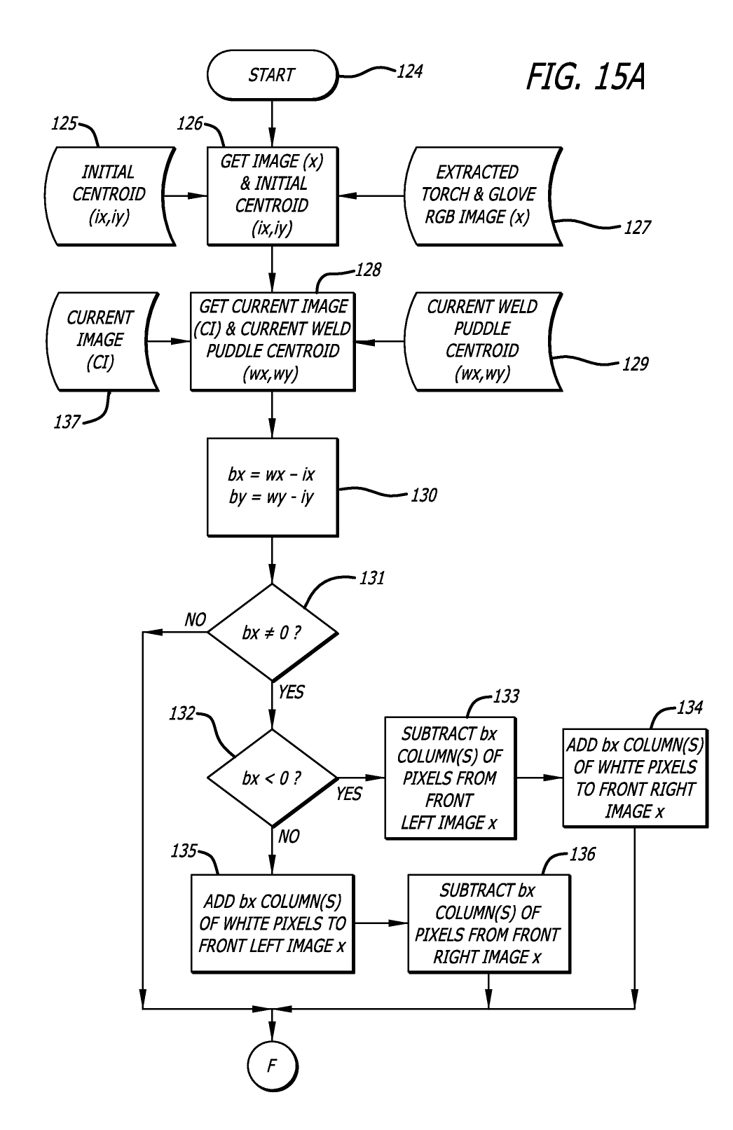

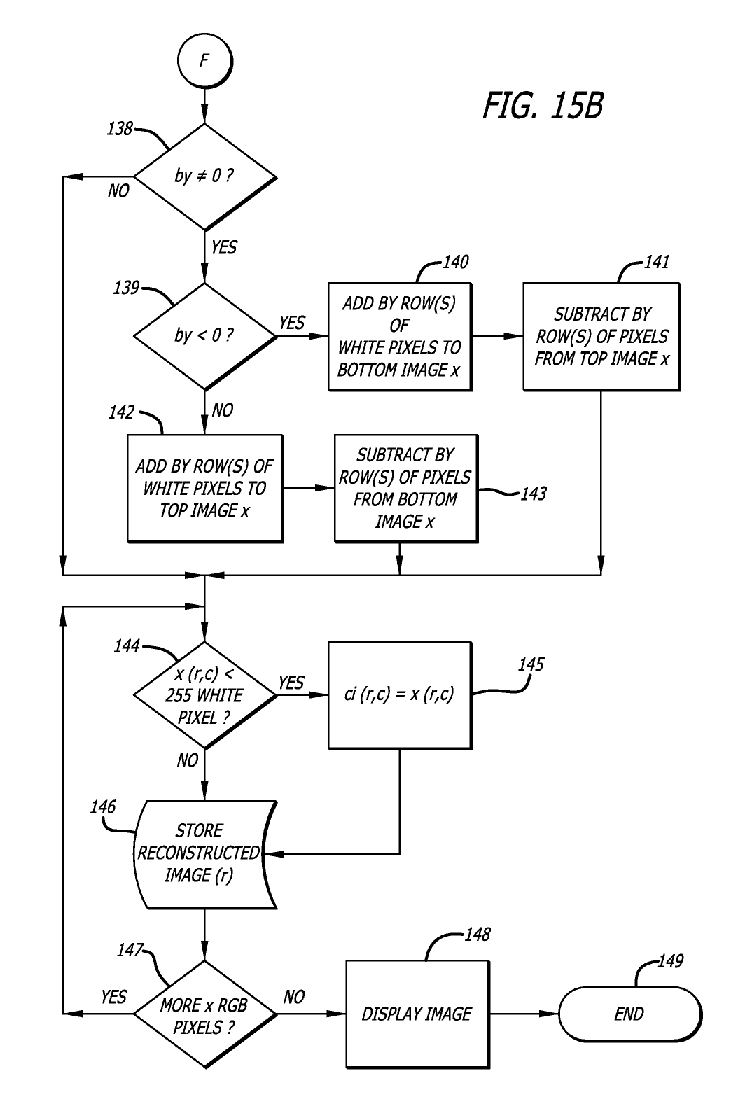

The acts used in producing a real-time mediated reality welding streaming video are depicted in FIGS. 15A and 15B. Starting in FIG. 15A at block 124, the extracted RGB torch and glove image (x) from block 127 and the initial centroid (ix, iy) from 125 are read from memory 36 and obtained by block 126. The current weld puddle vector (wx, wy) from block 129 is read from memory 36 and obtained by block 128. The current image (CI) from block 137 is read from memory 36 and obtained by block 128. An x-y coordinate (bx, by) value is calculated at block 130 that determines where the torch and glove should be placed on the current composited frame (CI). The calculation at block 130 subtracts the currently composited frame's x-y weld puddle vector from the initial x-y torch and glove vector, bx=wx−ix and by=wy−iy. These vectors are needed to adjust the torch and glove image so it can be inserted into the currently composited frame (CI). The column adjustment of the extracted torch and glove image begins at block 131. If at block 131 it is determined that bx equals zero, the column doesn't need processing 131 and column adjustment of the torch and glove image completes and the processing continues to FIG. 15B. If at block 131 it is determined that bx is not equal to zero, then the column needs to be adjusted. The type of adjustment is determined at block 132. If at block 132 it is determined that bx is less than zero, bx columns of pixels are subtracted from the front left torch and glove reference image x at block 133 and bx columns of white pixels are added to the front right image x at block 134 ensuring the adjusted torch and glove image size is the same as the original image size. Otherwise, at block 135 bx columns of white pixels are added to the front left image x and at block 136 bx columns of pixels are subtracted from the front right torch and glove reference image x. The column adjustment of the torch and glove image then completes and the processing continues to FIG. 15B.

The row adjustment of the extracted torch and glove image begins in FIG. 15B at block 138. If at block 138 it is determined that by equals zero, the row doesn't need processing and row adjustment of the torch and glove image completes and processing continues to block 144. If at block 138 it is determined that by is not equal to zero, the row needs to be adjusted. The type of adjustment is determined at block 139. If at block 139 it is determined that by is less than zero, by rows of white pixels are added to the bottom of image x at block 140 and by rows of pixels are subtracted from the top of the torch and glove reference image x at block 141. Otherwise, by rows of white pixels are added to top of image x at block 142 and by rows of pixels are subtracted from the bottom of the torch and glove reference image x at block 143. The row adjustment of the torch and glove image then completes and the processing continues to block 144.

The adjusted torch and glove RGB image is placed back onto the current composited image (ci) starting at block 144. The pixels of both images (x, ci) are read by row (r) and column (c). If at block 144 it is determined that the current pixel of the adjusted torch and glove image x is not a white pixel, the pixel from the torch glove image is substituted for the pixel on the currently composited image (ci) using the formula ci (r, c)=x (r, c) at block 145 and the resulting pixel r is stored in memory 36 at block 146. Otherwise, if at block 144 it is determined that the current pixel of the adjusted torch and glove image x is a white pixel, no pixel substitution is necessary and the current composited pixel ci is stored in memory 36 at block 146. If at block 147 it is determined that there are more pixels to be processed, the algorithm continues at block 144; otherwise the mediated reality video frame is displayed to the operator on the display screen 19 at block 148 and the process ends at block 149 and awaits for the next composited image frame (CI). It will be appreciated by one of ordinary skill in the art that techniques such as video inpainting, texture synthesis, matting, etc., could be used in the preceding algorithm (FIGS. 15A and 15B) to accomplish the same result.

FIGS. 7A, 7B, 8, 9, 11A, 11B, 13A, 13B, 13C, 15A and 15B are executed in real-time for each camera (or image sensor) frame in order to display streaming video on a frame-by-frame basis.

The various elements of the different embodiments may be used interchangeably without deviating from the present invention. Moreover, other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for altering visual perception during a welding operation, comprising:
obtaining a current image;
determining a background reference image;
determining a foreground reference image;
processing the current image by:
combining the current image and the background reference image; and
substituting the foreground reference image onto the combined image; and
displaying a processed current image.

2. A welding helmet comprising:
a mask; and
a mediated reality welding cartridge attached to the mask, the mediated reality welding cartridge including an image sensor and a display screen, and being configured to obtain a current image from the image sensor; determine a background reference image; determine a foreground reference image; process the current image by combining the current image and the background reference image, and substitute the foreground reference image onto the combine image; and display a processed image on the display screen.

3. A mediated reality welding cartridge for use with a welding helmet, the mediated reality welding cartridge comprising:
an image sensor;
a display screen;
a processor;
memory in the form of a non-transitory computer readable medium; and
a computer software program stored in the memory, which, when executed using the processor enables the mediated reality welding cartridge to obtain a current image from the image sensor; determine a background reference image; determine a foreground reference image; process the current image by combining the current image and the background reference image, and substitute the foreground reference image onto the combine image; and display a processed image on the display screen.
